(19) United States
(12) Patent Application Publication
(10) Pub. No.: US 2006/0147564 A1
(43) Pub. Date: Jul. 6, 2006

(54) Composition for preventing or treating allergic disease using black rice extract and its therapeutic use

(75) Inventor: Jae-Hong Kim, Gyeonggi-do (KR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: CELLMICS CO. LTD, Seoul (KR)

(21) Appl. No.: 10/551,820

(22) PCT Filed: Mar. 30, 2004
(86) PCT No.: PCT/KR04/00722

§ 371 Date: Feb. 10, 2006

(30) Foreign Application Priority Data
Apr. 4, 2003 (KR) 10-2003-0021476

(51) Int. Cl. A61K 31/353; A61K 36/899

(52) U.S. Cl. 424/750; 514/456

(57) ABSTRACT

The present invention relates to a composition for preventing or treating allergic diseases using black rice extract and its therapeutic use. More precisely, the present invention relates to a composition for preventing or treating allergic diseases comprising pelargonidin, cyanidin glycoside, or black rice extract including pelargonidin and cyanidin glycoside, which inhibit eosinophile accumulation in tissues, as an effective ingredient and a therapeutic use thereof. Pelargonidin, cyanidin glycoside or black rice extract including pelargonidin and cyanidin glycoside inhibit the accumulation of eosinophile in tissues and allergic inflammations thereby, so that they can be effectively used for preventing or treating allergic diseases associated with inflammation and eosinophile accumulation, such as allergic rhinitis, allergic conjunctivitis, asthma, chronic obstructive pulmonary disease, atopic dermatitis and allergic diarrhea, etc.

### FIELD OF THE INVENTION

The present invention relates to a composition for preventing or treating allergic diseases using black rice extract and its therapeutic use. More precisely, the present invention relates to a composition for preventing or treating allergic diseases comprising pelargonidin, cyanidin glycoside, or black rice extract including pelargonidin and cyanidin glycoside as an effective ingredient and a therapeutic use thereof.

### BACKGROUND

In general, an allergic disease is known to be caused by allergic inflammation in airway or tissues such as bronchus etc. In particular, an allergic disease develops as follows: Allergens (antigens) such as dust, pollen, fungi, various foods and drugs etc, come into an individual through the respiratory organs, the digestive organs or skin and then combine with IgE antibodies attached on mast cell surface in a tissue. Then, the mast cells secrete histamine. Histamine, the most important chemical mediator causing an allergic reaction in nasal mucosa, causes edema in nasal mucosa by increasing vascular permeability and induces primary allergic responses such as tears, nose drippings and pruritis etc, by stimulating sensory nerve terminal. In addition to histamine, chemical mediators with chemotaxis such as eosinophilic chemotactic factor and leukotriene are secreted from mast cells in tissues. Eeosinophiles are moved to an allergic development region (chemotaxis) by a chemotactic factor, causing late allergic responses, such as tissue injury, inflammatory response and hypersensitivity, etc.

Asthma, allergic rhinitis and atopic dermatitis are the examples of allergic diseases, which keep increasing as air pollution by soot etc, become serious. Yet, any effective therapeutic agent for satisfactory treatment of the allergic diseases has not been developed. Once treatment stopped, symptoms recur in a few days or weeks, requiring improvements in safety and effectiveness of conventional treatment agents.

As of today, the major therapeutic agents for treating allergic diseases are corticosteroids just relieving symptoms, which are not only far from the fundamental treatment of the disease by removing a cause but also carry serious side effects (Rabe K F, et. al., Eur Respir J Suppl., 34:34s-40s, 2001). Most conventional therapeutic agents for treating allergic diseases have only a function of inhibiting histamine, so that they cannot inhibit late responses by the accumulation of eosinophiles in tissues, which is a major reason for inflammation, resulting in chronic allergic symptoms. Therefore, it is an urgent and important demand to develop a novel anti-allergic medicine overcoming the problems of conventional therapeutic agents for treating allergic diseases.

Black rice (Oryza sativa L.), a rice including much anthocyanins, is a health food including calcium, vitamin, niacin etc, much more than white rice. Black rice has been known to have effects of improving the regulating homeostatic function of human body and enhancing the immune function. Besides, black rice has been known to have effects of preventing diseases, anti-oxidation, anticancer and in particular lowering cholesterol.

Anthocyanins are pigmental glycosides found in red parts of flowers or fruit peel. The anthocyanins are compounds in which a specific hydroxyl group of glucose is linked to a functional group of alcohol, phenol, aldehyde, etc, by ether bond. More than 200 anthocyanins including delphinidin, cyanidin, pelargonidin, peonidin and malvidin have been found so far. Anthocyanins are involved in anti-inflammatory action, antimicrobial activity, and lowering cholesterol, and especially have a 5-7 fold higher anti-oxidation activity than tocopherol, a natural anti-oxidant (Tedesco I, et. al., J. Nutr. Biochem., (9):505-511, 2001; Youdim K A, et. al., Biochim. Biophys. Acta., 1523(1):117-122, 2000). However, the concrete effect of each compound of anthocyanins has not explained yet.

### DETAILED DESCRIPTION OF THE INVENTION

The present inventors have made studies to develop a therapeutic agent for treating allergic diseases effectively by inhibiting inflammation due to eosinophiles, which is one of late responses of allergic diseases. As a result, they confirmed that black rice extract, among many other Chinese medicines and natural substances, can effectively inhibit asthma, one of the representative allergic diseases. Moreover, the present inventors completed this invention by confirming that anthocyanins included in black rice extract, in particular, pelargonidin and cyanidin glycoside, inhibit the accumulation of eosinophiles and inflammation in tissues, and thereby can treat allergic diseases including asthma.

It is an object of this invention to provide a method for preventing or treating allergic diseases using black rice extract.

It is also an object of this invention to provide a novel therapeutic use of black rice extract.

It is a further object of this invention to provide a method for preventing or treating allergic diseases using pelargonidin or cyanidin glycoside.

It is another object of this invention to provide a method for inhibiting the accumulation of eosinophiles in cells, tissues or a body using pelargonidin or cyanidin glycoside.

It is also an object of this invention to provide a novel therapeutic use of pelargonidin or cyanidin glycoside.

It is a further object of this invention to provide a composition for preventing or treating allergic diseases comprising one or more selected from a group consisting of black rice extract, pelargonidin and cyanidin glycoside.

In order to achieve the object above, the present invention provides a method for preventing or treating allergic diseases, comprising administering an effective amount of black rice extract, pelargonidin or cyanidin glycoside to an individual in need thereof.

To achieve another object of the invention, the present invention provides a method for inhibiting the accumulation of eosinophiles in cells, in tissues or a body, comprising administering pelargonidin or cyanidin glycoside to an individual in need thereof.

To achieve another object of the invention, the present invention provides a use of black rice extract, pelargonidin or cyanidin glycoside for the preparation of a therapeutic agent for preventing or treating allergic diseases.

Further to achieve another object of the invention, the present invention provides a use of pelargonidin or cyanidin glycoside for the preparation of a therapeutic agent for inhibiting the accumulation of eosinophiles.

The present invention will be described below.

The present invention provides a composition for preventing or treating allergic diseases comprising black rice extract.

The present invention also provides a composition for preventing or treating allergic diseases comprising pelargonidin (3,5,7-trihydroxy-2-(4-hydroxyphenyl)-1-benzopyrylium chloride) represented by Formula 1, pharmaceutically acceptable salts or glycosides thereof, as an effective ingredient.

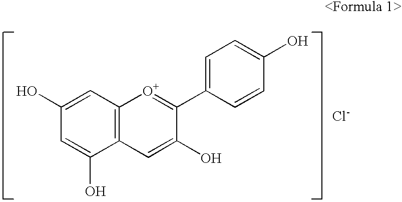

The present invention also provides a composition for preventing or treating allergic diseases comprising cyanidin glycoside (cyanidin 3-O-β-glucopyranoside) represented by Formula 2 or pharmaceutically acceptable salts thereof as an effective ingredient.

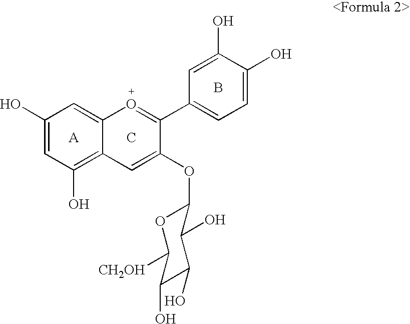

The present invention also provides a composition for preventing or treating allergic diseases comprising one or more selected from a group consisting of black rice extract, pelargonidin or cyanidin glycoside as an effective ingredient.

Black rice extract of the present invention includes pelargonidin (3,5,7-trihydroxy-2-(4-hydroxyphenyl)-1-benzopyrylium chloride) represented by Formula 1 or cyanidin glycoside (cyanidin 3-O-β-glucopyranoside) represented by Formula 2.

Black rice (Oryza sativa L.) in this invention is rice colored in black, includes much anthocyanins, and is commercially available with ease.

Black rice extract included in a composition for preventing or treating allergic diseases of the present invention can be prepared from Oryza sativa L. according to a conventional extraction methods well known in the pertinent art. The extraction methods included, without limitation, alcohol extraction, water extraction, organic solvent extraction and supercritical fluid extraction, etc. Preferably, one of water and organic solvents like C₁-C₄ lower alcohols, acetone, methyl acetate, ethyl acetate, glycerol, propylene glycol, 1,3-butylene glycol, n-hexane, diethyl ether, benzene and methylene chloride, or a mixture thereof can be used. Black rice is pulverized, to which one of the above solvent is added. Remnants are discarded by filtering. The filtered solution is concentrated using a vacuum evaporator by stirring. Solvent is removed and the concentrated solution is freeze-dried, making it pulverized.

The preferable extraction temperature is 15˜80° C., and more preferably 25˜60° C. The extraction time depends on the extraction temperature, but generally 5˜24 hours, and preferably 7˜12 hours. If a shaker is used for extraction, the extraction efficiency can be increased.

In an embodiment of the present invention, ethanol was added to black rice, leading to extraction at 35° C. for 7 hours. Then, the extracted solution was evaporated by drying, resulting in powdered black rice extract (see Example 1).

Pelargonidin represented by Formula 1, included in a composition for preventing or treating allergic diseases of the present invention, is believed to have a strong anti-oxidative activity, even though its concrete mechanism of action has not been disclosed yet. In particular, pelargonidin is less toxic but better absorbed, making it a prominent therapeutic agent suitable for administration to human (Ross J A, et. al., Annu Rev Nutr., 2002; 22:19-34. Review). Nevertheless, no other use except as an anti-oxidative agent has been known so far.

Cyanidin glycoside (cyanidin 3-O-β-glucopyranoside) represented by Formula 2, included in a composition for preventing or treating allergic diseases of the present invention, is a natural substance belonging to anthocyanins and was reported to have a strong anti-oxidative activity.

Pelargonidin shown by Formula 1 and Cyanidin glycoside shown by Formula 2, which are included in a composition for preventing or treating allergic diseases of the present invention, can either be purchased or be prepared by a conventional synthetic method (Nakajima N, et al. Biosci. Biotechnol. Biochem., 61(11):1926-1928, 1997, Amorini A M, et al. Free Radic. Res., 35(6):953-966). Preferably, they can be separated and purified from natural substances. It is more preferred to separate them from black rice. Pelargonidin or cyanidin glycoside can be separated and purified from black rice by a conventional method well known in the pertinent art. Particularly, the effective ingredients of black rice can be extracted using water or organic solvents. Then, chromatography is performed to separate and purify ingredients, thereby obtaining pure target compounds.

In an embodiment of the present invention, asthma was induced in mice by sensitizing with ovalbumin, in which inhibition of inflammation, a general symptom of an allergic disease, by black rice extract was investigated. As a result, black rice extract of the present invention inhibited inflammation remarkably in lungs of the mice with asthma induced by ovalbumin (see FIG. 1).

In another embodiment of the present invention, it was investigated which anthocyanins, major components of black rice extract, could inhibit inflammation and the accumulation of eosinophiles in tissues, general symptoms of allergic diseases. Major anthocyanins, such as pelargonidin, delphinidin, peonidin and cyanidin glycoside were administered to mice with asthma induced by ovalbumin. As a result, it was confirmed that pelargonidin and cyanidin glycoside inhibited the accumulation of eosinophiles, inflammation inducing cells, in airway and inflammation in lung remarkably (see FIG. 2-FIG. 4, Table 1). Delphinidin among many anthocyanins had no effects. Peonidin showed slight effect. But, Pelargonidin and cyanidin glycoside were confirmed to inhibit the accumulation of eosinophiles in airway and inflammation in lung, suggesting that pelargonidin and cyanidin glycoside could be effectively used as a composition for preventing or treating allergic diseases.

Therefore, a composition comprising black rice extract, pelargonidin or cyanidin glycoside of the present invention can be effectively used for preventing or treating an allergic disease selected from a group consisting of bronchial asthma, chronic obstructive pulmonary disease, hay fever, vasomotor rhinitis, hypertrophic rhinitis, allergic bronchitis, transient pulmonary infiltration, allergic gastritis, allergic diarrhea, allergic stomatitis, intestinal purpura, periarteritis nodosa, occlusive endarteritis, angina pectoris, endocarditis, urticaria, angioneurotic edema, erythema nodosum, purpura, atopic dermatitis, phlycten, sympathetic ophthalmia, allergic conjunctivitis and allergic keratitis in mammals, in particular, humans.

An ‘effective amount’ in this invention means the amount of a compound or an extract showing a preventive or a treating effect when administered to a patient. Generally, black rice extract can be administered in an amount of 1-100 mg/kg a day and preferably 10-30 mg/kg a day. Pelargonidin represented by Formula 1 can be administered in an amount of 0.1-10 mg/kg a day and preferably 0.5-2 mg/kg a day. Cyanidin glycoside represented by Formula 2 can be administered in an amount of 1-30 mg/kg a day and preferably 5-20 mg/kg a day. The compounds and extracts can be administered either once or several times a day within a possible effective amount. The extent of administered amount of black rice extract, pelargonidin or cyanidin glycoside may vary suitably by the administration route, the subject of administration, age, sex, weight, the degree of a disease and other individual differences of a patient. A composition comprising black rice extract, pelargonidin or cyanidin glycoside of the present invention is not limited to the dosage form, administration route or method, as far as it retains the inventive effects.

An ‘individual’ herein means mammals including human being. The individual include a patient in need of treatment.

A composition of the present invention can be prepared and administered in many forms and by various methods. For example, any of oral, rectal, local, intraperitoneal, intraocular, intrapulmonary and intranasal administration is possible. And, the composition can be formulated into various dosage forms, such as tablet, troche, dispersant, suspension, liquid preparation, capsule, cream, ointment and aerosol.

A composition of the present invention comprising pelargonidin includes pelargonidin and its pharmaceutically acceptable salts or glycosides thereof as an active ingredient, and may further include pharmaceutically acceptable carriers and other therapeutic components. A composition of the present invention comprising cyanidin glycoside includes cyanidin glycoside and its pharmaceutically acceptable salts, and additionally may include pharmaceutically acceptable carriers and other therapeutic components. The ‘pharmaceutically acceptable salt’ herein means a salt prepared from a pharmaceutically acceptable nontoxic base or acid (inorganic base or inorganic acid and organic base or organic acid are included).

Compositions for oral, rectal, local, hypodermic, parenteral including intramuscular and intravenous, intraocular, intrapulmonary (nasal inhalation or oral inhalation) or intranasal administration are all included and the most suitable administration route is selected from the above according to characteristics and severity of a disease and characteristics of active ingredients. A composition can be conveniently prepared by a single dosage form following a common preparation method well known in the field of pharmaceutics.

As for inhalation, compounds or extract of the present invention are conveyed as an aerosol spray using a pressurized pack or a sprayer. Compounds or extract of the present invention are also conveyed as a powder form, which can be inhaled through an aeration powder inhalation device. A preferable conveying system for inhalation is measuring dosage inhalation (MDI) aerosol, which can be formulated in the form of solution or suspension by mixing one of propellants, such as fluorocarbon or hydrocarbon with black rice extract or compounds of Formula 1 and Formula 2.

Transdermal preparation, aerosol, cream, ointment, lotion and spray are good examples for the local administration formulation of black rice extract, pelargonidin or cyanidin glycoside.

As an active ingredient, black rice extract, pelargonidin or cyanidin glycoside can be mixed with pharmaceutically acceptable carriers by a general pharmaceutical technique for practical administration. A carrier may vary according to the administration route (for example, oral or parenteral administration comprising intravenous administration). For the preparation of liquid formulations for oral administration, such as suspensions, elixirs and solutions, general pharmaceutical excipients such as water, glycol, oil, alcohol, flavoring agents, antiseptics and coloring agents can be used. Solid formulations for oral administration include powders, capsules and tablets. These solid formulations are prepared by mixing one or more suitable excipients, such as starch, glucose, microcrystalline cellulose, diluents, granulating agents, lubricants, binding agents and disintegrating agents, etc. Solid formulations are preferable to liquid formulations for oral administration. Tablets and capsules are the most convenient forms for oral administration, for which solid pharmaceutical carriers are used. If required, tablets can be coated according to standard aqueous technique or non-aqueous technique. Carriers for parenteral administration include water, suitable oil, saline, water-soluble glucose or glycol, etc. And stabilizers and preservatives may be additionally included. The antioxidants, such as Sodium bisulfite, sodium sulfite and ascorbic acid are suitable for stabilizers. Benzalconium chloride, methyl- or propyl-paraben and chlorobutanol are the examples for preservatives. Other pharmaceutically acceptable carriers listed in the following documents are also available (Remington's Pharmaceutical Sciences, 19th ed., Mack Publishing Company, Easton, Pa., 1995).

The above black rice extract, pelargonidin or cyanidin glycoside can be provided in the form of a food composition for preventing and treating allergic diseases. A food composition of the invention includes all possible types, such as functional food, nutritional supplements, health food and food additives, etc. Such food composition can be prepared in various forms according to conventional methods informed well in the pertinent art. For example, as health food, an extract of the invention itself can be taken either in the form of tea, juice and drink or in the form of granule, capsule and powder.

For the production of functional food, an extract or compounds of the present invention can be added to beverages (comprising alcoholic beverages), fruits and their processed food (ex: canned fruits, bottled food, jam, marmalade, etc), fish, meat and its processed food (ex: ham, sausages, corned beef, etc), bread and noodles (wheat noodles, buckwheat noodles, ramyun, spaghetti, macaroni, etc), fruit juices, various drinks, cookies, wheat gluten, dairy products (ex: butter, cheese, etc), vegetable oil, margarine, vegetable protein, retort food, frozen food and various seasonings (ex: soybean paste, soy sauce, sauce, etc), etc.

In order to be used as a food additive, an extract or compounds of the present invention are preferably prepared in the form of powder or concentrate.

A preferable content of an extract or compounds of the present invention in a food composition of the invention is 1˜90 weight % out of total weight of the composition. 10˜50 weight % is more preferable. As explained above, black rice extract, pelargonidin or cyanidin glycoside inhibits the accumulation of eosinophiles, inflammation inducing cells, and inflammation in tissues, so that health food composition comprising the above can effectively be used as a subsidiary for preventing or treating allergic diseases.

The present invention further provides a therapeutic use of black rice extract, pelargonidin or cyanidin glycoside. Particularly, the present invention provides a use of pelargonidin or cyanidin glycoside for the preparation of a therapeutic agent for inhibiting the accumulation of eosinophiles in cells, tissues or an individual in need thereof. The therapeutic agent may further include pharmaceutically acceptable carriers in addition to pelargonidin or cyanidin glycoside. Pharmaceutically acceptable carriers have been exemplified above.

The present invention also provides a use of black rice extract, pelargonidin or cyanidin glycoside for the preparation of a therapeutic agent for preventing and treating allergic diseases. Allergic diseases have been exemplified above.

### BRIEF DESCRIPTION OF THE DRAWINGS

FIG. 1 is a set of microphotographs showing the inhibition of inflammation in lung of mice, after treating black rice extract to the mice with asthma induced by ovalbumin.

A: Wild-type mice,

B: Mice with asthma induced by ovalbumin,

C: Mice treated with black rice extract (10 mg/kg)

FIG. 2 is a graph showing each inhibition rates of eosinophile accumulation in airway of mice, after treating the anthocyanins to the mice with asthma induced by ovalbumin.

1: Wild-type mice,

2: Mice with asthma induced by ovalbumin,

3: Mice treated with Pelargonidin (0.5 mg/kg),

4: Mice treated with Pelargonidin (1.25 mg/kg),

5: Mice treated with Peonidin (0.5 mg/kg),

6: Mice treated with Peonidin (1.25 mg/kg),

7: Mice treated with Delphinidin (0.5 mg/kg),

8: Mice treated with Delphinidin (1.25 mg/kg)

FIG. 3 is a set of microphotographs showing the inhibition of inflammation in lung of mice, after treating pelargonidin to the mice with asthma induced by ovalbumin.

A: Wild-type mice

B: Mice with asthma induced by ovalbumin,

C: Mice treated with Pelargonidin (1.25 mg/kg),

D: Mice treated with Delphinidin (1.25 mg/kg)

FIG. 4 is a set of microphotographs showing the inhibition of inflammation in lungs of mice, after treating cyanidin glycoside to the mice with asthma induced by ovalbumin.

A: Wild-type mice

B: Mice with asthma induced by ovalbumin,

C: Mice treated with Cyanidin glycoside (1.5 mg/kg),

D: Mice treated with Cyanidin glycoside (4.5 mg/kg)

### EXAMPLES

The present invention will be described by the following examples in more detail. However, the examples shown below are provided solely to illustrate the invention; the scope of the invention should not be construed to be limited thereto.

### Example 1

### Preparation of Black Rice Extract

Black rice (domestic production, Kyungdong market, Seoul, Korea) was pulverized, to which 100% ethanol was added, followed by extraction at 35° C. for 7 hours. The obtained extract was evaporated and remnants were freeze-dried, resulting in powder type black rice extract.

### Example 2

### Examination of Asthma-Inhibiting Effect of Black Rice Extract

In this example, experiments were performed to investigate whether black rice extract could inhibit allergic asthma by inhibiting inflammation in mice with asthma induced by ovalbumin.

At first, in order to prepare animal models of asthma, 200 μl of ovalbumin solution (ovalbumin 200 μg and alumina gel 1000 μg were dissolved in physiological saline) was injected in each abdominal cavity of twenty 10-week old female mice (C57BL/6, Damul Science, Daejeon, Korea). Two weeks later, 200 μl of ovalbumin solution(2% w/v) was sprayed on each mouse to sensitize it. 200 μl of 1% ovalbumin solution was sprayed again on the 21^{st}, the 22^{nd} and the 23^{rd} day, and 10% ovalbumin solution was sprayed again on the 25^{th} day to sensitize.

The above mice with asthma induced by sensitizing with 10% ovalbumin were divided into two groups; one was used as a negative control group treated with nothing and the other was treated with 10 mg/kg of black rice extract. Black rice extract was administered by intraperitoneal injection and the injection was performed twice on the 24^{th} and the 25^{th} day from the treatment of ovalbumin.

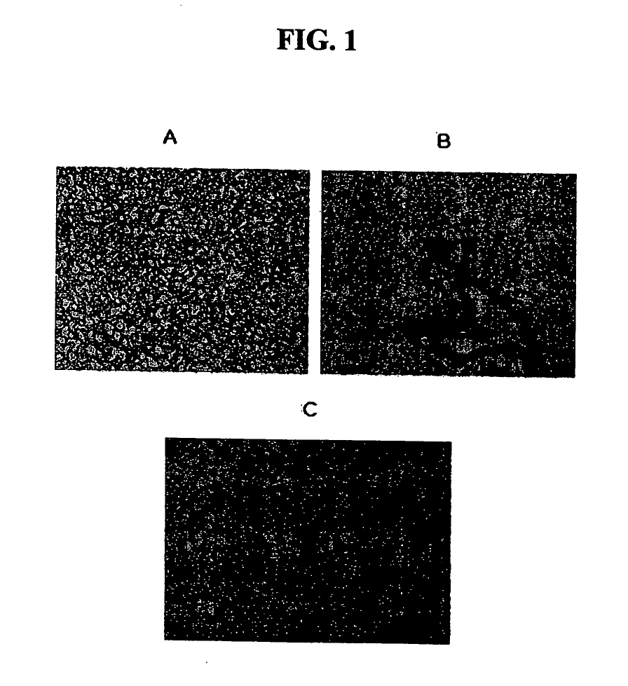

48 hours after the treatment of black rice extract, mice were sacrificed using ether. Inflammation in lung of each mouse was investigated. As a result, as shown in FIG. 1, inflammation in bronchi of mice with asthma induced by ovalbumin, an asthma-inducing antigen, was greatly increased (see FIG. 1B), comparing wild-type normal mice (see FIG. 1A). However, the inflammation in bronchi was remarkably decreased by the administration of black rice extract (see FIG. 1C). Therefore, it was confirmed that black rice extract could inhibit asthma effectively.

### Example 3

### Effect of Pelargonidin Included in Black Rice Extract on the Accumulation of Eosinophiles in Airway

Major components of black rice extract having the above asthma-inhibiting effect are anthocyanins. In this example, experiments were performed to investigate whether the anthocyanins, major active components of black rice extract, could inhibit asthma, and exactly which anthocyanines could inhibit asthma.

Mice with asthma induced by the same method used in the above Example 2 were divided into four groups; one was used as a negative control, and three other groups were used as experimental groups and treated with peonidin, delphinidin and pelargonidin (0.5 mg/kg, 1.25 mg/kg), respectively. Each compound was administered by intraperitoneal injection and the injection was performed twice on the 24^{th} and the 25^{th} day from the treatment of ovalbumin.

On the second day from the treatment, each mouse was sacrificed using ether. Then, a microtube was connected to trachea. PBS (phosphate-buffered saline, 0.8 ml) was injected and recovered through the microtube, which was repeated twice, resulting in the obtainment of bronchoalveolar lavage fluid (BALF). The fluid was centrifuged to separate cells in airway lumen and various proteins secreted from the cells and lung.

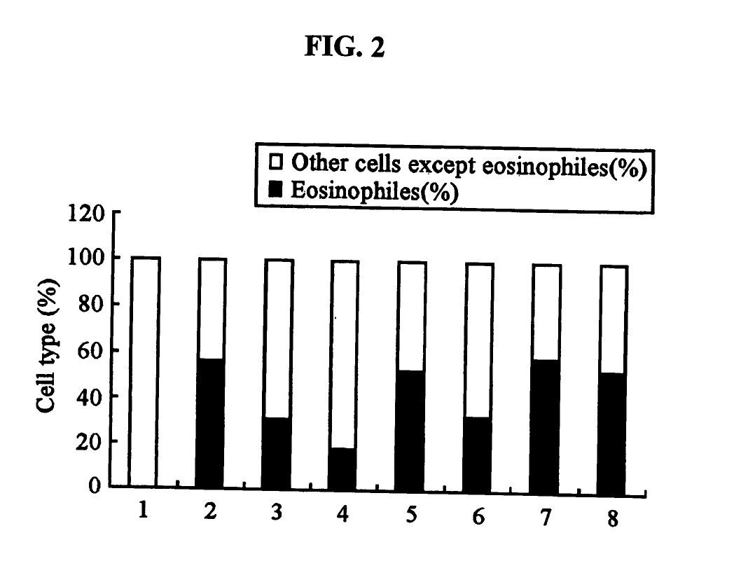

The separated cells were fixed on a slide using cytospin and stained with Diff-Quick staining solution. Photographs were taken by a digital camera attached on Carzeiss microscope (model: AXIOVERT 25-CEL). 5 random regions per each sample were picked to count eosinophiles, and the percentage of eosinophiles in each sample was shown in FIG. 2.

As shown in FIG. 2, the percentage of eosinophiles in airway of mice exposed on ovalbumin was 58%. On the contrary, the accumulation of eosinophiles in airway was inhibited in all the groups treated with anthocyanines, such as pelargonidin, peonidin and delphinidin. In particular, in the case of administering pelargonidin by 0.5 mg/kg and 1.25 mg/kg, respectively, the percentage of eosinophiles in airway was decreased to 30% and 20% each. It was confirmed that pelargonidin could inhibit more effectively the accumulation of eosinophiles in airway than other anthocyanines.

### Example 4

### Inhibitory Effect of Pelargonidin on Inflammation in Asthma

In this example, experiments were performed to investigate whether pelargonidin, previously proved to inhibit the accumulation of eosinophiles greatly in airway, could inhibit allergic asthma by inhibiting inflammation in lung.

Mice with asthma induced by 10% ovalbumin, just like in the above Example 2, were divided into three groups; group 1 was used as a negative control treated with nothing, group 2 was used as a positive control treated with 1.25 mg/kg of delphinidin and group 3 was treated with 1.25 mg/kg of pelargonidin. Each compound was administered by intraperitoneal injection and the injection was performed twice on the 24^{th} and the 25^{th} day from the treatment of ovalbumin.

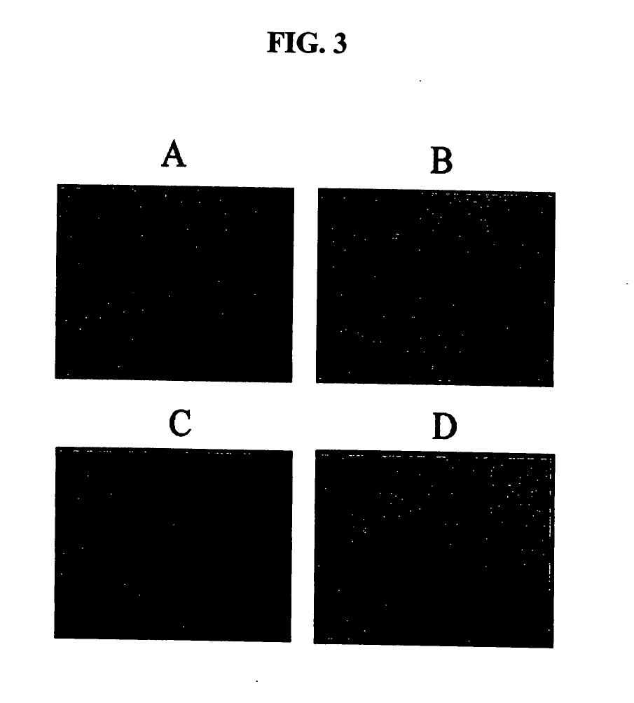

48 hours after the treatment of each compound, mice were sacrificed using ether and inflammation in lung of each mouse was investigated. As a result, as shown in FIG. 3, inflammation in bronchi of mice was remarkably increased by ovalbumin (FIG. 3B), an asthma-inducing antigen, compared with that in normal wild-type mice (FIG. 3A). On the other hand, inflammation was remarkably reduced by the administration of pelargonidin (FIG. 3C). The inflammation-inhibiting effect of pelargonidin was outstanding in both groups treated with 1.25 mg/kg of pelargonidin and treated with 0.5 mg/kg of pelargonidin (data not shown). On the contrary, inflammation in bronchi was not much inhibited by delphinidin (1.25 mg/kg), another anthocyanin (FIG. 3D).

### Example 5

### Inhibitory Effect of Cyanidin Glycoside Included in Black Rice Extract on the Accumulation of Eosinophiles in Airway and Inflammation in Asthma

In this example, the effect of cyanidin glycoside included in black rice extract on the accumulation of eosinophiles in airway and the asthma inhibiting capability thereof were investigated.

In order to prepare animal models with asthma, 0.5 ml of ovalbumin solution (500 μg of ovalbumin and 10 mg of alumina gel were dissolved in 1 ml of PBS) was injected into abdominal cavities of twenty 5-week old female mice(Balb/c, Orient, Seoul) on first and 10^{th} experiment day. The ovalbumin solution was sprayed on each mouse on the 21^{st}, the 22^{nd} and the 23^{rd} day to induce asthma.

Mice (n=15) with asthma induced by ovalbumin were divided into three groups; one was used as a negative control treated with nothing and two other groups were used as experimental groups each treated with cyanidin glycoside by 1.5 mg/kg and 4.5 mg/kg, respectively. Cyanidin glycoside was orally administered once a day in succession from the 2^{nd} day to the 23^{rd} day.

24 hours after final sensitization, each mouse was sacrificed using ether and a microtube was connected into trachea. 0.8 ml of PBS was injected and recovered through the microtube, which was repeated twice. The obtained bronchoalveolar lavage fluid (BALF) was centrifuged to separate cells in airway lumen and various proteins secreted from the cells and lung.

The separated cells were fixed on a slide using cytospin and stained with Diff-Quick staining solution. Photographs were taken by a digital camera attached on Carzeiss microscope (model: AXIOVERT 25-CEL). 5 random regions per each sample were picked to count eosinophiles, and the percentage of eosinophiles in each sample was represented in Table 1.

TABLE 1

| | | No. of | Percentage of |
|---|---|---|---|
| | Concentration | animals | eosinophiles |
| wild type mice | — | 5 | 0.1 ± 0.0 |
| group | | | |
| Negative control | — | 5 | 62.7 ± 4.3 |
| group | | | |
| Cyanidin glycoside | 1.5 mg/kg | 5 | 49.5 ± 5.8* |
| treated group | | | |
| Cyanidin glycoside | 4.5 mg/kg | 5 | 38.2 ± 6.2* |
| treated group | | | |

*Significant difference (p < 0.05)

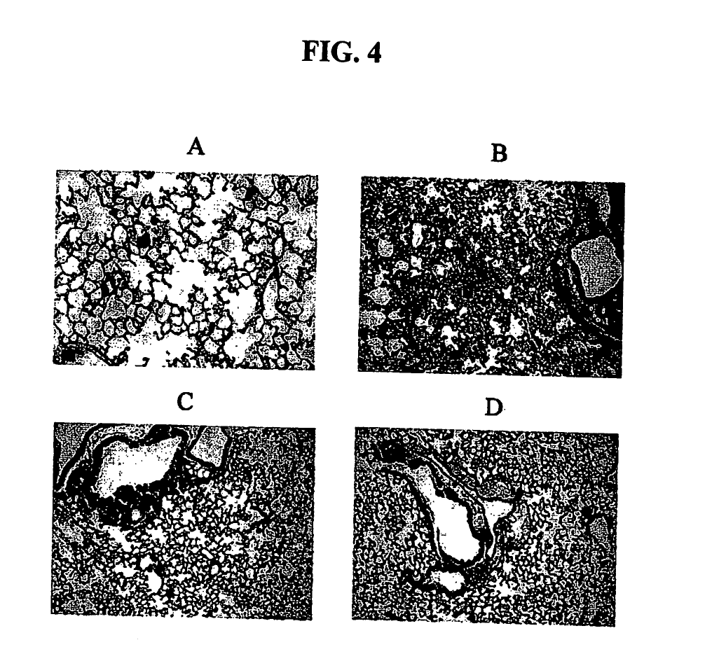

As shown in Table 1, the percentage of eosinophiles in airway of mice exposed on ovalbumin (negative control) was 63% high, but the accumulation of eosinophiles in airway was proved to be inhibited by the administration of cyanidin glycoside. Precisely, as cyanidin glycoside concentration increased, the percentage of eosinophiles in airway decreased to 49% and 38% each; compared with a negative control. It was confirmed that the accumulation of eosinophiles in airway was effectively inhibited by cyanidin glycoside. Inflammation in lung cells of the sacrificed mice was also investigated and the result was represented in FIG. 4. As shown in FIG. 4, inflammation in bronchi of mice was remarkably increased by ovalbumin (FIG. 4B), an asthma-inducing antigen, compared with that of normal wild-type mice (FIG. 4A). However, the inflammation was remarkably reduced by the administration of cyanidin glycoside (FIG. 4C and FIG. 4D).

### Example 6

### Production of a Beverage Composition Comprising Black Rice Extract of the Present Invention

A beverage composition was prepared by mixing black rice extract (25%) obtained in the above Example 1, vitamin A (0.15%), vitamin D (0.2%), vitamin B₂ (0.15%), vitamin C (2.0%), taurine (3.0%), polydextrose (2.5%) and purified water together.

### INDUSTRIAL APPLICABILITY

As described in the above, pelargonidin and cyanidin glycoside or black rice extract including pelargonidin and cyanidin glycoside was proved to inhibit the accumulation of eosinophiles in tissues and allergic inflammation caused thereby. Therefore, pelargonidin, cyanidin glycoside or black rice extract of the present invention can effectively be used for preventing or treating allergic diseases accompanying inflammation and the accumulation of eosinophiles in tissues, for example, allergic rhinitis, allergic conjunctivitis, asthma, chronic obstructive pulmonary disease, atopic dermatitis and allergic diarrhea, etc.

1. A method for preventing or treating allergic diseases, comprising administering an effective amount of black rice extract to an individual in need thereof.

2. The method of claim 1, wherein the black rice extract comprises pelargonidin represented by Formula 1 or cyanidin glycoside represented by Formula 2.

3. A method for preventing or treating allergic diseases, comprising administering an effective amount of pelargonidin represented by Formula 1, pharmaceutically acceptable salts or glycosides thereof to an individual in need thereof.

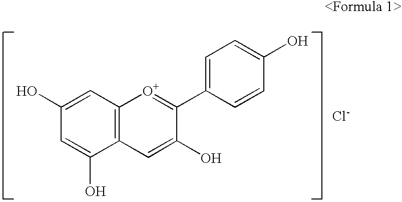

4. A method for preventing or treating allergic diseases, comprising administering an effective amount of cyanidin glycoside (cyanidin 3-O-β-glucopyranoside) represented by Formula 2 or pharmaceutically acceptable salts thereof to an individual in need thereof.

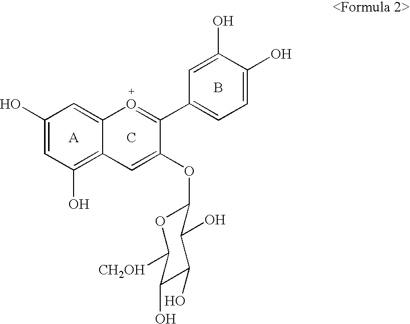

5. A method for inhibiting the accumulation of eosinophiles in cells, tissues or a body, comprising administering pelargonidin represented by Formula 1, pharmaceutically acceptable salts or glycosides thereof to an individual in need thereof.

6. A method for inhibiting the accumulation of eosinophiles in cells, tissues or a body, comprising administering cyanidin glycoside represented by Formula 2 or pharmaceutically acceptable salts thereof to an individual in need thereof.

7. The method of claim 1, wherein the allergic disease is selected from a group consisting of bronchial asthma, chronic obstructive pulmonary disease, hay fever, vasomotor rhinitis, hypertrophic rhinitis, allergic bronchitis, transient pulmonary infiltration, allergic gastritis, allergic diarrhea, allergic stomatitis, intestinal purpura, periarteritis nodosa, occlusive endarteritis, angina pectoris, endocarditis, urticaria, angioneurotic edema, erythema nodosum, purpura, atopic dermatitis, phlycten, sympathetic ophthalmia, allergic conjunctivitis and allergic keratitis.

8. The method of claim 7, wherein the allergic disease is bronchial asthma or chronic obstructive pulmonary disease.

9. A composition for preventing or treating allergic diseases comprising one or more selected from a group consisting of black rice extract, pelargonidin represented by Formula 1 or cyanidin glycoside represented by Formula 2.

10. A method for the preparation of a therapeutic agent for preventing or treating allergic diseases comprising using black rice extract.

11. A method for the preparation of a therapeutic agent for preventing or treating allergic diseases comprising using pelargonidon, pharmaceutically acceptable salts or glycosides thereof.

12. A method for the preparation of a therapeutic agent for preventing or treating allergic diseases comprising using cyaniding 3-Oβ-glucopyranside).

13. The method of claim 10, wherein the allergic disease is selected form a group consisting of bronchial asthma, chronic obstructive pulmonary disease, hay fever, vasomotor rhinitis, hypertrophic rhinitis, allergic bronchitis, transient pulmonary infiltration, allergic gastritis, allergic diarrhea, allergic stomatitis, intestinal purpura, periarteritis nodosa, occlusive endarteritis, angina pectoris, endocarditis, urticaria, angioneurotic edema, erythema nodosum, purpura, atopic dermatitis, phlycten, sympathetic ophthalmia, allergic conjunctivitis and allergic keratitis.

14. A method for the preparation of a therapeutic agent for inhibiting the accumulation of eosinophiles in cells, tissues or an individual in need thereof comprising using pelargonidin represented by Formula 1, Pharmaceutically acceptable salts or glycosides thereof.

15. A method for the preparation of a therapeutic agent for inhibiting the accumulation of eosinophiles in cells, tissues or individual in need thereof comprising using cyaniding glycoside represented by Formula 2 or pharmaceutically acceptable salts thereof.
